(19) United States
(12) Patent Application Publication
(10) Pub. No.: US 2004/0042962 A1
(43) Pub. Date: Mar. 4, 2004

(54) Method and apparatus for nonmagnetic identification of organisms, and a method of use of the same

(76) Inventor: Marcus Pfister, Erlangen (DE)

Correspondence Address: HARNESS, DICKEY & PIERCE, P.L.C., P.O.BOX 8910, RESTON, VA, 20195, US

(21) Appl. No.: 10/621,402

(22) Filed: Jul. 18, 2003

(30) Foreign Application Priority Data
Jul. 18, 2002 (DE) 102 32 682.7

(51) Int. Cl. A61K 49/00; C12Q 1/04

(52) U.S. Cl. 424/9.6; 435/34

(57) ABSTRACT

For the purpose of more accurate and more rapid nonmagnetic identification of organisms by way of a marking code on a marking element that can be provided on the organism, use is made of a marking code. The marking code is composed of regions with numerous different optically detectable and machine evaluatable criteria. In this case, annular marking elements made from non-magnetizable material are preferred.

The present application hereby claims priority under 35 U.S.C. §119 on European patent application number DE 10232682.7 filed Jul. 18, 2002, the entire contents of which are hereby incorporated herein by reference.

### FIELD OF THE INVENTION

The invention generally relates to a method and an apparatus for identifying organisms, and generally relates to the use of the method and/or of the apparatus.

### BACKGROUND OF THE INVENTION

A method for animal identification is already known (WO 87/04900). In this case, the marking element includes a tubelet that can be implanted in the organism and is equipped with elements to achieve a transponder action so that the marking elements stored, for example, on a microchip, can be interrogated from outside by electromagnetic waves. However, such apparatuses are not suitable in some animal imaging methods, such as the application of magnetic resonance (MR), since in this case the marking code can easily be changed and the result of identification is falsified.

It is also generally known, moreover, for organisms, chiefly animals, to be ringed with marking elements that have as marking code series of numbers or letters that are optically detected by the human eye and input manually into an evaluation device. However, reading and input errors often occur in the case of this method, and so there is a lack of accuracy in identification. Moreover, this method requires human participation. The identification also lasts a relatively long time in this case.

Appropriate methods are also customary in the case of metal ear clips that are provided on the ear of the organism, for example the cow or sheep. The same problems arise in the case of these markings and also in the case of the written markings applied directly to organisms such as mice.

### SUMMARY OF THE INVENTION

It is an object of an embodiment of the invention to accelerate a method and/or an apparatus for nonmagnetic identification of organisms, and to attain even more accurate animal identification in conjunction with a largely machine-based design. The achievement of this object is important chiefly in the case of dynamic observations of progress of experimental animals in biological, medical and pharmaceutical research, where nonambiguity and speed are of the essence.

Preferred embodiments of the invention are explained in greater detail below with the aid of the drawings and the descriptive text belonging thereto.

Use is made, in an embodiment of the invention, of a marking code that includes, in particular, numerous regions with, in particular, a plurality of different optically detectable and machine evaluatable criteria. This is in contrast to so-called bar codes, in the case of which numerous strip-shaped regions are certainly present, wherein use is made of only two different optically detectable criteria, such as bright and dark. Such criteria can be, for example, gray-scale values, colors and/or fluorescence intensities of the regions of the marking element which form the marking code. Thus, the so-called “gray-scale values” are determined by the intensity of the reflection, absorption, refraction and/or diffraction of light at the surface of the marking element regions. In other words: a comparison is made of the respective brightness of the relevant regions with one another. If the color of the respective region is to serve as criterion for the marking code, the wavelengths of the electromagnetic waves reflected, diffracted or refracted at the surface of the regions are compared with one another. Here, the expression “optically detectable” is to be understood in the widest sense: it includes not only the light visible to humans, but also extends to wavelength regions, such as IR and UV wavelength regions, that can certainly be detected by an optical sensor but not by the human eye.

The regions are preferably in the shape of strips or rings, but can also have other configurations in two-dimensional and three-dimensional extent.

It is preferred to use sensors in the form of a CCD camera for the optical detection.

Encoding by different gray-scale values is to be recommended. In order to facilitate the detection, a specific encoding should always include all the different brightnesses exactly once in each case. This can be performed, for example, by a single permutation of the brightnesses and the assignment of precisely one permutation to a specific identification number. Thus,

N!=1×2×3× . . . (N−1)×N

encodings are possible with N brightness. With seven brightnesses, that is to say N=7, the result is N!=5040 different encodings. With eight gray-scale values, the number increases to 40320 encodings.

An advantage of such encoding resides in this case in that, on the one hand, the regions or fields of different brightnesses mutually delimit themselves (since two fields of equal brightness can never be situated contiguously). On the other hand the brightness values need not be exactly determined but rather the N brightness values need only be ordered.

According to another alternative embodiment of the invention, N-valued number systems can be encoded from N brightnesses: with N brightnesses and m places, it is therefore possible to encode N^{m} numbers, wherein N and m are integers. For m=N, 823543 encoding numbers would result in the case of seven brightnesses, and as many as 16777218 would result with eight brightnesses. However, this alternative complicates the evaluation, or requires additional delimitations between the individual regions or fields (since in this case juxtaposed places of equal brightness cannot be excluded), and this influences the production or size of the overall identification.

At least four, even better at least six, different criteria, such as brightness values, should be distributed over the regions. However, all brightness encodings are conceivable in principle, specifically preferably those which mutually delimit themselves and can be evaluated as robustly as possible.

During identification, the optical detection and evaluation by machine, that is to say the optical determination by machine, of the marking code is preferably performed fully automatically. However, the detection of the marking element as such can also take place partially semi-automatically by, for example, an operator using the computer mouse to draw a region that is as constricted as possible, for example a rectangle, over the part of the image of the organism in which the marking element is expected. The remainder of the process of finding the marking element and the marking code can then goes ahead automatically. However, it is preferred to carry out fully automatic detection and evaluation in the case of which image processing software independently detects the marking element and its marking code on the basis of characteristic features such as edges or homogeneity patterns.

The evaluation is also expediently performed fully automatically. In this case, use is preferably made for the purpose of encoding of the “edge” brightness gradient between neighboring regions. This is strongly expressed at the borders of the regions or encoding fields, but is, by contrast, expressed rather weakly and non-directionally in the background image. Further, it is even almost not expressed at all within the encoding fields themselves. It is recommended to carry out after this determination a smoothing of the signals and then a binarization of the gray-scale values.

It is, moreover, expedient when coherent, homogeneous foreground regions obtained therefrom are combined to form fixed units. Efficient processing of binary images is expediently performed by run length encoding, as a result of which coherent foreground regions can be calculated and detected very quickly. In some circumstances, these need to be post-processed, for example subsequently smoothed, separated and combined, in order to conform with specific geometrical considerations and to remove possible interference from components of the background noise or image noise.

Details on this are explained in further detail with the aid of the drawing.

The regions or “encoding fields” used for the marking codes can, however, also be separated from one another by other image processing methods. Suitable for this purpose is segmentation in the case of which predeterminable image data properties of the segments are used to segment the detected image data, coherent regions are formed by way of an assignment of the segments with the aid of predeterminable assignment criteria, coherent regions are filtered and, finally, coherent regions are analyzed with the aid of predeterminable analyzing criteria. More precise details on this are described in the literature. The segmentation of the image data can be carried out, in particular, by applying the watershed algorithm, by means of region growing or by binarization.

It is particularly preferred to use annular marking elements as carriers of the marking code. This is because, in the case of a ring, the marking codes are visible virtually from all perspectives, as is also further shown diagrammatically with the aid of the drawing.

It is advisable to use a nonmagnetizable material, in particular one made from plastic such as PVC, for the marking element. This is done in order also to be able to apply those animal imaging methods in which electromagnetic waves or else strong magnetic fields occur, as in the case of magnetic resonance (MR).

The annular marking element include, for example, individual rings, bonded to one another, of different brightness values or other different optically perceptible criteria for the individual regions. However, it is also possible to use a single compact ring that can be provided with different regions capable of being perceived optically by printing or other machine-based processing. The encoding regions should in this case, however, likewise run annularly and preferably outside and inside, in order to facilitate the optical detectability from as many directions as possible independently of position and rotation of the ring.

An embodiment of the invention can be used with particular preference for the purpose of nonmagnetic animal identification in the case of in vivo small animal imaging. Particularly in biological, medical and pharmaceutical research, it is primarily small animals that are provided with newly developed active components or drugs, whereupon it is possible either by new imaging methods, for example using light in the NIR region, but also with the aid of classical technologies such as MR, CD, PET or SPECT and by optical fluorescence imaging to determine changes, owing to inflammations or tumors, for example, in specific regions of the animal. The observation of the development and temporal change, for example, with the administration of the drug to be tested then permits conclusions to be drawn on the effectiveness of the drug. An embodiment of the invention permits fast and accurate identification of the relevant animal, and this also contributes to carrying out the series of tests very quickly and accurately, as a result of which the development of drugs and testing can be accelerated in a relatively simple way.

### BRIEF DESCRIPTION OF THE DRAWINGS

Exemplary embodiments of the invention are explained in more detail below with the aid of the drawing, in which:

FIG. 1 shows a workflow of the identification method during a small animal experiment in accordance with the invention, with only a single manual “manipulation” of the marking of a region on the image;

FIG. 2 shows a scale of 6 different “gray levels”, that is to say of 6 substantially rectangular regions of different light reflection from 0 to 100%;

FIG. 3 shows three different positions of annular marking elements that in each case have four annular regions with in each case different, that is to say overall four different brightness values, with the gray levels 0, 30, 70 and 100% reflection of the incident light;

FIG. 4 shows annular regions found by software evaluation in a specific image segment, and correspondingly automatically determined gray-scale values, specifically in

a. with a strip of the gray level encoding illustrated schematically in FIG. 2,

b. as a result of the image preprocessing with the aid of an edge recognition method and subsequent binarization method, and

c. after further processing of the annular fields found to form coherent components, after which the respective mean gray-scale value of the components is determined, and

FIG. 5 shows regions or fields and their determined gray-scale values, found by the evaluation controlled by way of software, specifically

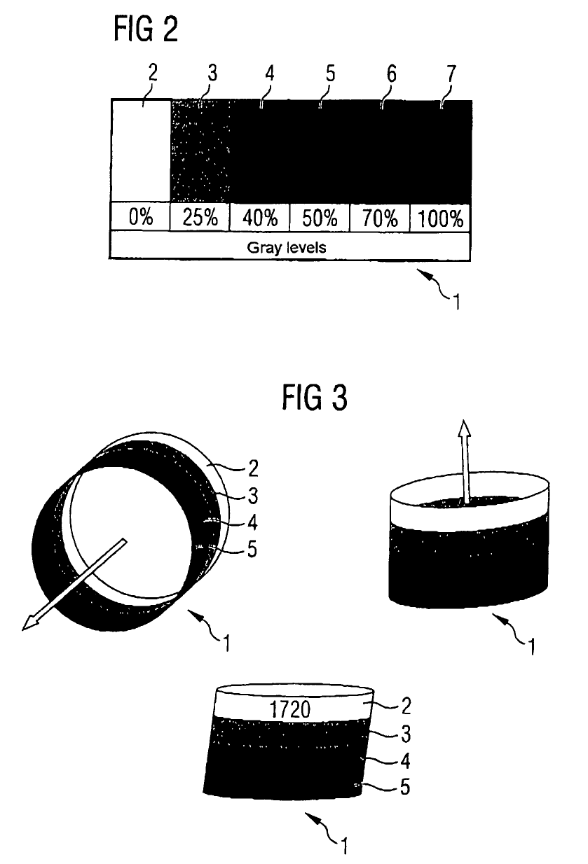

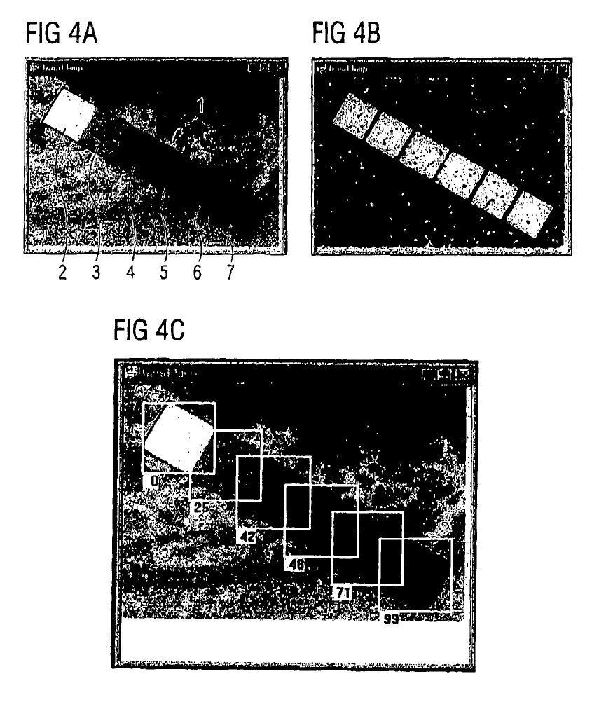

a. and b. with further examples of the methods, illustrated schematically in FIGS. 2 and 4, with the difference that by contrast with FIG. 4 gray-scale value arrangements have been permuted and the strip orientation has been changed.

### DETAILED DESCRIPTION OF THE PREFERRED EMBODIMENTS

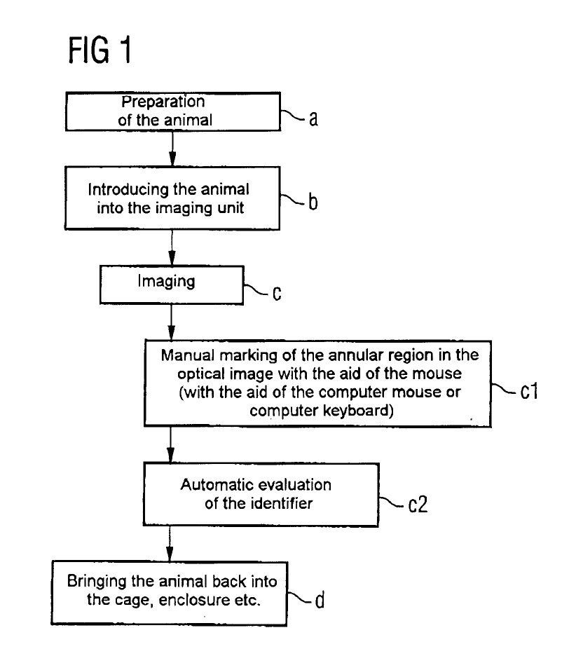

In accordance with the workflow or flowchart of FIG. 1, according to the method according to an embodiment of the invention, the animal to be examined is removed from the cage in step a and prepared for the examination, for example by anesthesia. Subsequently, the animal is brought in step b into the imaging position in the imaging and identifying unit, whereupon the imaging and identification takes place in step c. For this purpose, in step c1 the operator uses the keyboard or computer mouse in order to mark the “annular region” manually in the optical image in the case of the use of an annular marking element.

The automatic evaluation of the regions of the marking element detected by the CCD camera then takes place in step c2 with the aid of its marking codes, whereupon the animal is brought back again into the cage in step d, for example after awakening from the anesthesia.

The method according to an embodiment of the invention and the apparatus according to an embodiment of the invention ensure that the animal identification is performed not only quickly, but also extremely exactly. As such, there is no risk that anomalies in an animal are assigned to a different animal and the entire series of examinations (examination of a multiplicity of animals) is falsified.

In accordance with FIG. 2, a marking element 1 is assigned six strip-shaped fields or regions (2 . . . 7) each having different gray levels of 0%, 25%, 40%, 50%, 70% and 100% absorption. As a result, the encoding differs from conventional bar codes, in the case of which a plurality of regions are likewise juxtaposed in a strip-shaped fashion, but only two different gray levels, in particular 0% and 100%, alternate with one another and, for example, the spacing or the width of the fields is used for encoding purposes. An embodiment of the invention therefore differs in principle from such bar codes through the greater number of different criteria of the optical characteristics of the relevant regions of the marking element.

In accordance with FIG. 3, the marking element 1 is of annular design and—as already mentioned above—respectively has four regions (2 . . . 5) with different brightnesses or gray levels in each case. Here, the different criteria are present not only on the outside of the ring, but also on the inner cylindrical surface of the ring, and so all the “brightness codes” can be optically detected from many different observation perspectives independently of the position and arrangement of the annular marking element. The marking element with the regions 2, 3, 4 and 5 of different brightnesses or gray levels consists of nonmagnetic material, in particular plastic, and so MR examinations of the animal are also possible without a problem.

The illustrations of FIG. 4 are based for the purpose of detecting the identification or marking code on a semiautomatic method: the operator firstly uses the keyboard or the computer mouse to draw as constricted as possible a rectangle on the image representation, whereupon the detection of the regions or fields 2, 3, 4, 5, 6, 7 of the annular marking element 1 is performed automatically within this rectangle. The result of this is then an image corresponding to FIG. 2 with the individual regions 2 to 7 of the different gray levels. In image a, the marking code of FIG. 2 is copied into a background strongly affected by noise, in order to simulate an image segment from a realistic laboratory picture. In order to worsen the image, noise was additionally added to the result and it was given a soft focus.

The result of the image preprocessing is illustrated schematically in image b. The annular fields are fashioned and separated from one another with the aid of edge recognition methods and a subsequent binarization method.

The annular fields found have been combined in image c to form coherent components, and the respective mean gray-scale value of the components has been determined. The rectangular borders with the numbers 0, 25, 42, 48, 71 and 99 below them indicate only the rectangular contour of the component but not the rectangle itself in which the gray-scale value is determined. In fact, use is made for this purpose only of the surfaces found in accordance with figure b, independently of their orientation.

It becomes clear from this that the automatic evaluation approaches the actual gray-scale values of FIG. 2 up to 1%, and to this extent operates very accurately in order to exclude instances of mistaken identity and therefore an inaccurate measurement result. Of course, correction methods and other methods of improvement can also be applied in this case through the inclusion of known geometries. Should encodings be used which permit neighboring encoding fields of identical brightness (without dark parting lines), artificial separations then have to be undertaken in any case by the processing and evaluation software with the aid of spacing considerations.

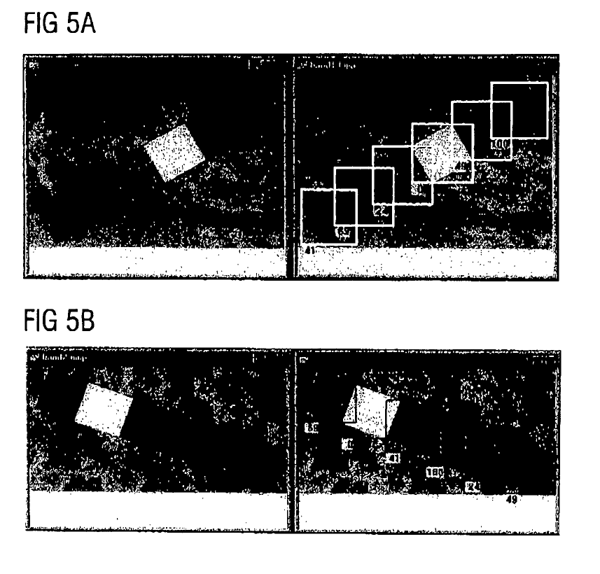

Further examples roughly in accordance with the explanations relating to FIG. 4 are shown in FIG. 5 in the component images a and b; there are six regions of different gray levels in each case for the arrangement of regions of the marking code shown schematically in FIG. 2. In accordance with FIG. 5, as well, the gray-scale values determined correspond up to 1% with those selected according to FIG. 2, specifically independently of orientation and pattern of gray-scale values of the strip.

The invention being thus described, it will be obvious that the same may be varied in many ways. For example, the marking element including the marking code can be placed on a label, with the label thereafter being placed on the organism. Such and other variations are not to be regarded as a departure from the spirit and scope of the invention, and all such modifications as would be obvious to one skilled in the art are intended to be included within the scope of the following claims.

What is claimed is:

1. A method for nonmagnetic identification of an organism, comprising:
optically detecting a marking code of a marking element on the organism to be examined; and,
evaluating the detected marking code to identify the organism carrying the marking element, wherein the marking code includes a plurality of regions including at least three different optically detectable and machine evaluatable criteria.

2. The method as claimed in claim 1, wherein the optically detectable and machine evaluatable criterion include intensity of at least one of reflection, absorption, diffraction and refraction of light.

3. The method as claimed in claim 1, wherein the optically and machine evaluatable criterion include at least one of wavelength of at least one of reflection, absorption and diffraction of light, color and fluorescence of regions of the marking element.

4. The method as claimed in claim 1, wherein the differences in at least one of the intensity of at least one of the reflection, absorption, diffraction and brightness of light, wavelength thereof, color and fluorescence at the borders of respectively neighboring regions of the marking element is used as the marking code.

5. The method as claimed in claim 1, wherein the regions are encoded by a plurality of N different brightnesses.

6. The method as claimed in claim 1, wherein at least one of the regions and region boundaries are encoded by N^{m} numbers, wherein N and m are integers.

7. The method as claimed in claim 1, wherein the optical determination and machine evaluation of the marking element is performed at least semi-automatically, and wherein the optical determination and machine evaluation of the marking code is performed fully automatically.

8. The method as claimed in claim 7, wherein, for the purpose of fully automatic determination, the edge gradients of neighboring regions of the marking element are optically determined.

9. The method as claimed in claim 8, wherein foreground regions belonging to at least one of a component and a criterion are combined to form fixed units.

10. The method as claimed in claim 9, wherein the combination is undertaken by run length encoding.

11. The method as claimed in claim 1, wherein image data are segmented into segments with the aid of predeterminable image data properties, wherein coherent regions are formed with the aid of an assignment of the segments with the aid of predeterminable assignment criteria, wherein coherent regions are filtered, and wherein coherent regions are at least one of analyzed and evaluated with the aid of predeterminable analytical criteria.

12. The method as claimed in claim 11, wherein the segmentation of the image data is carried out with the aid of the watershed algorithm.

13. The method as claimed in claim 11, wherein the segmentation of the image data is carried out by region growing.

14. The method as claimed in claim 11, wherein the segmentation of the image data is carried out by binarization.

15. The method as claimed in claim 1, wherein a CCD camera is used for optically detecting the marking code.

16. An apparatus for identifying an organism including a marking element, comprising:
a sensor for optically detecting a marking code of the marking element; and
an evaluation device for evaluating the detected optical signal, wherein the marking element is an annular marking element including at least one marking code, the marking code including a plurality of regions with optically detectable and machine evaluatable criteria.

17. The apparatus as claimed in claim 16, wherein the annular marking element includes a nonmagnetizable material.

18. The apparatus as claimed in claim 17, wherein the annular marking element includes plastic.

19. A method for in vivo small animal imaging identification, using the apparatus of claim 16.

20. The method as claimed in claim 1, wherein the optically detectable and machine evaluatable criterion include brightness of regions of the marking element as the marking code.

21. The method as claimed in claim 2, wherein the differences in at least one of the intensity of the reflection, absorption, diffraction and brightness of light and the wavelength thereof, the color and fluorescence at the borders of respectively neighboring regions of the marking element is used as marking code.

22. The method as claimed in claim 3, wherein the differences in at least one of the intensity of the reflection, absorption, diffraction and brightness of light and the wavelength thereof, the color and fluorescence at the borders of respectively neighboring regions of the marking element is used as marking code.

23. The apparatus of claim 16, wherein the marking code includes a plurality of regions including at least three different optically detectable and machine evaluatable criteria.

24. The apparatus as claimed in claim 17, wherein the annular marking element includes PVC.

25. The method of claim 1, for in vivo small animal imaging identification.

26. An apparatus for identifying an organism including a marking element, comprising:
sensing means for optically detecting a marking code of the marking element; and
evaluation means for evaluating the detected optical signal, wherein the marking element is an annular marking element including at least one marking code, the marking code including a plurality of regions with optically detectable and machine evaluatable criteria.

27. The apparatus as claimed in claim 26, wherein the annular marking element includes a nonmagnetizable material.

28. The apparatus as claimed in claim 27, wherein the annular marking element includes plastic.

29. The apparatus of claim 26, wherein the marking code includes a plurality of regions including at least three different optically detectable and machine evaluatable criteria.

30. The apparatus as claimed in claim 27, wherein the annular marking element includes PVC.

31. A marking element for placement on and identification of an organism, comprising:
at least one optically detectable marking code, wherein the marking element is an annular marking element for placement on the organism and wherein the marking code includes a plurality of regions with optically detectable and machine evaluatable criteria.

32. The marking element of claim 31, wherein the marking code includes a plurality of regions including at least three different optically detectable and machine evaluatable criteria.

33. A label including the marking element of claim 31.

34. A method for nonmagnetic identification of an organism, comprising:
placing a marking element, including an optically detectable marking code, on an organism to be examined, wherein the detectable marking code is evaluatable to identify the organism, and wherein the marking code includes a plurality of regions including at least three different optically detectable and evaluatable criteria.

35. The method as claimed in claim 34, wherein the optically detectable and evaluatable criteria include intensity of at least one of reflection, absorption, diffraction and refraction of light.

36. The method as claimed in claim 34, wherein the optically and evaluatable criteria include at least one of wavelength of at least one of reflection, absorption and diffraction of light, color and fluorescence of regions of the marking element.

37. The method as claimed in claim 34, wherein the differences in at least one of the intensity of at least one of the reflection, absorption, diffraction and brightness of light, wavelength thereof, color and fluorescence at the borders of respectively neighboring regions of the marking element is used as the marking code.

38. The method as claimed in claim 34, wherein the regions are encoded by a plurality of N different brightnesses.

39. The method as claimed in claim 34, wherein at least one of the regions and region boundaries are encoded by N^{m} numbers, wherein N and m are integers.
